(19) United States
(12) Patent Application Publication
(10) Pub. No.: US 2021/0139525 A1
(43) Pub. Date: May 13, 2021

(54) NUCLEOTIDE PRODRUGS

(71) Applicant: CERECOR, INC., Rockville, MD (US)

(72) Inventors: Stephen B. Thomas, New York, NY (US); Patrick J. Crutcher, New York, NY (US); Fabrizio Pertusati, Cardiff (GB); Elisa Pileggi, Cardiff (GB)

(21) Appl. No.: 16/959,901

(22) PCT Filed: Jan. 7, 2019
(86) PCT No.: PCT/US19/12560

§ 371 Date: Jul. 2, 2020

Related U.S. Application Data
(60) Provisional application No. 62/613,892, filed on Jan. 5, 2018.

(51) Int. Cl. C07H 19/20; C07H 19/10

(52) U.S. Cl. CPC C07H 19/20; C07H 19/10

(57) ABSTRACT

The invention relates to nucleotide prodrugs and pharmaceutical preparations thereof. The invention further relates using the prodrugs of the invention in the treatment of mitochondrial DNA (mtDNA) depletion syndrome (MDS).

### FIELD OF THE INVENTION

This relates generally to purine radicals with the saccharide radical esterified by phosphoric or polyphosphoric acids.

### BACKGROUND OF THE INVENTION

Mitochondrial DNA (mtDNA) depletion syndrome (MDS) encompasses a group of genetic disorders characterized by a severe reduction in mtDNA content leading to respiratory chain deficiency in affected tissues and organs. MDS arises due to defects in mtDNA maintenance caused by mutations in nuclear genes that function in either mitochondrial nucleotide synthesis, deoxyribonucleoside triphosphate (dNTP) metabolism or mtDNA replication. There are also some MDSs with unknown pathophysiology.

Some exemplary MDSs are deoxyguanosine kinase (DGUOK) deficiency, thymidine kinase 2 (TK2) deficiency, mitochondrial neurogastrointestinal encephalomyopathy (MNGIE), mitochondrial DNA polymerase (POLG) deficiencies (including Alpers-Huttenlocher syndrome, SANDO syndrome, MIRAS, etc.), MPV17-related hepatocerebral and RRM2B-related myopathies. Of known mutations, there are over ten genes that have been linked to MDS (TK2, DGUOK, POLG, MPV17, RRM2B, SUCLA2, SUCLG1, TYMP, C10orf2, and SAMHD1).

Direct supplementation with nucleosides, deoxyribonucleoside monophosphates (dNMPs), deoxyribonucleoside diphosphates (dNDPs) or dNTPs has shown the ability to rescue mtDNA depletion in in vitro models of MDS and increase overall survival in animal models of MDS in vivo. However, the pharmacological prospects for nucleosides, dNMPs, dNDPs and dNTPs as practical treatments for MDS in humans are low. The negatively charged phosphates on dNMPs, dNDPs and dNTPs preclude diffusion across cellular membranes. Furthermore, intra- and extracellular phosphatases effectively dephosphorylate dNMPs, dNDPs and dNTPs to the base nucleoside prior to reaching the desired site of action. Although the base nucleoside can enter the cell via passive and active transport mechanisms, it cannot by itself address the deficiencies of MDS given that phosphorylation of a nucleoside to a dNMP is the rate-limiting step of nucleotide synthesis and, in many cases, MDS patients lack the enzyme responsible for this transformation. Such considerations require high doses of nucleosides, dNMPs, dNDPs or dNTPs to potentially achieve therapeutic benefit.

Thus, there is a need for new therapies for MDS, and in particular for therapies that can effectively provide dNMPs, dNDPs or dNTPs to mitochondria.

### SUMMARY OF THE INVENTION

In a first embodiment, the invention provides compounds having the structure of formula I:

[chemical structure]

and pharmaceutically acceptable salts and prodrugs thereof, wherein:
R¹ is aryl or heteroaryl;
R² and R^{2′}, each independently, are hydrogen, alkyl or aralkyl;
R³ is alkyl or aralkyl;
R⁴ is hydrogen or alkyl; or R² and R⁴ together with the —C—N— moiety that separates them forms a heterocycle; and
NT is selected from a nucleobase or nucleobase prodrug moiety.

In a second embodiment, the compounds of formula I include the exemplary compounds depicted in Table I.

In a third embodiment, the invention provides compounds having the structure of formula II:

[chemical structure]

and pharmaceutically acceptable salts and prodrugs thereof, wherein:
R¹ is hydrogen or alkyl;
R^{2a} and R^{2b}, each independently, are hydrogen, alkyl or aralkyl, or a natural amino acid side chain;
R³ is alkyl; and
NT is a nucleobase or a nucleobase prodrug moiety.

In a fourth embodiment, the invention provides pharmaceutical compositions of the subject compounds of formula I or formula II.

In a fifth embodiment, the invention provides methods of using these compounds or compositions in the treatment of MDSs such as deoxyguanosine kinase (DGUOK) deficiency, thymidine kinase 2 (TK2) deficiency, mitochondrial neurogastrointestinal encephalomyopathy (MNGIE), mitochondrial DNA polymerase (POLG) deficiencies (including Alpers-Huttenlocher syndrome, SANDO syndrome, MIRAS, etc.), MPV17-related hepatocerebral myopathy, or RRM2B-related myopathy; or in treating a mitochondrial DNA depletion syndrome linked to a mutation in TK2, DGUOK, POLG, MPV17, RRM2B, SUCLA2, SUCLG1, TYMP, C10orf2, or SAMHD1.

### BRIEF DESCRIPTION OF THE DRAWINGS

FIG. 1 shows the results of a study on the ability of certain compounds to rescue mtDNA depletion in patient-derived fibroblasts.

FIG. 2 shows the recovery of mtDNA copy number in DGUOK-deficient rat hepatocytes after administration of exemplary compounds.

### INDUSTRIAL APPLICABILITY

The invention relates to the use of a compound of formula I, formula Ia, formula II, or a compound selected from Table I, or a pharmaceutically acceptable salt thereof in the treatment of MDSs such as deoxyguanosine kinase (DGUOK) deficiency, thymidine kinase 2 (TK2) deficiency, mitochondrial neurogastrointestinal encephalomyopathy (MNGIE), mitochondrial DNA polymerase (POLG) deficiencies (including Alpers-Huttenlocher syndrome, SANDO syndrome, MIRAS, etc.), MPV17-related hepatocerebral myopathy, or RRM2B-related myopathy; or in treating a mitochondrial DNA depletion syndrome linked to a mutation in TK2, DGUOK, POLG, MPV17, RRM2B, SUCLA2, SUCLG1, TYMP, C10orf2, or SAMHD1.

The use involves the administering the compound of the invention to a patient in need thereof.

### BEST MODE FOR CARRYING OUT THE INVENTION

### Definitions

Chemistry terms used herein, unless otherwise defined herein, are used according to conventional usage in the art, as exemplified by The McGraw-Hill Dictionary of Chemical Terms, Parker S., ed. (McGraw-Hill, San Francisco, Calif., USA, 1985).

The term “acyl” refers to a group represented by the general formula hydrocarbylC(O)—, preferably alkylC(O)—.

“Administering” or “administration of” a compound or an agent to a patient or subject can be carried out using one of a variety of methods known to one skilled in the pharmaceutical art. A compound or an agent can be administered intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. The phrases “parenteral administration” and “administered parenterally” mean modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The term “agent” is used to denote a chemical compound (such as an organic compound or a mixture of chemical compounds.

The term “alkenyl” refers to an aliphatic group containing at least one double bond and is intended to include both “unsubstituted alkenyls” and “substituted alkenyls”.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, isopropyl (i-propyl), n-butyl, sec-butyl, tert-butyl, pentyl, hexyl, pentyl and octyl. A C1-C6 straight chained or branched alkyl group is also referred to as a “lower alkyl” group. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C1-6 alkyl, C3-6 cycloalkyl, halogen, carbonyl, cyano, or hydroxyl. In more preferred embodiments, the substituents on substituted alkyls are selected from fluoro, carbonyl, cyano, or hydroxyl. The moieties substituted on the hydrocarbon chain can themselves be substituted, if appropriate. For instance, the substituents of a substituted alkyl include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like. Exemplary substituted alkyls are described below. Cycloalkyls can be further substituted with alkyls, alkenyls, alkoxys, alkylthios, aminoalkyls, carbonyl-substituted alkyls, —CF3, —CN, and the like.

The term “alkynyl” refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents can occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The terms “amine” and “amino” are art-recognized and refer to both unsubstituted and substituted amines and salts thereof, e.g., a moiety that can be represented by:

[chemical structure]

wherein each RA independently represents a hydrogen or a hydrocarbyl group, or two RA are taken together with the N atom to which they are attached complete a heterocycle having from 4 to 8 atoms in the ring structure.

The term “aminoalkyl” refers to an alkyl group substituted with an amino group.

The term “aralkyl” refers to an alkyl group substituted with an aryl group.

The term “aryl” includes substituted or unsubstituted single-ring aromatic groups in which each atom of the ring is carbon. Preferably the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl can be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl can be selected from saturated, unsaturated and aromatic rings. A “cycloalkenyl” group is a cyclic hydrocarbon containing one or more double bonds.

The term “ester” refers to a group —C(O)ORA wherein RA represents a hydrocarbyl group.

The terms “halo-” and “halogen” means halogen and includes chloro-, fluoro-, bromo-, and iodo-.

The terms “hetaralkyl” and “heteroaralkyl” refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” means an atom of any element other than carbon or hydrogen.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “hydroxyalkyl” refers to an alkyl group substituted with a hydroxy group.

The term “lower” when used in conjunction with a chemical moiety is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “modulate” as used herein includes the inhibition or suppression of a function or activity as well as the enhancement of a function or activity.

The terms “patient,” “subject,” or “individual” are used interchangeably and refer to either a human or a non-human animal. These terms include mammals, such as humans, primates, livestock animals, companion animals and rodents.

The phrase “pharmaceutically acceptable” is art-recognized and refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” refers to an acid addition salt or a basic addition salt which is suitable for or compatible with the treatment of patients. The term includes any non-toxic organic or inorganic salt of any base compounds represented by formula I, formula Ia or formula II. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts can exist in either a hydrated, solvated or substantially anhydrous form. In general, the acid addition salts of compounds of formula I, formula Ia or formula II are more soluble in water and various hydrophilic organic solvents, and generally demonstrate higher melting points in comparison to their free base forms. The selection of the appropriate salt will be known to one skilled in the pharmaceutical art. Other non-pharmaceutically acceptable salts, e.g., oxalates, can be used, for example, in the isolation of compounds of formula I, formula Ia or formula II for laboratory use, or for subsequent conversion to a pharmaceutically acceptable acid addition salt. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to one skilled in the pharmaceutical art.

The phrase “pharmaceutically acceptable carrier” means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “prodrug” refers to a compound that is metabolized, for example hydrolyzed or oxidized, in the host after administration to form the compound of the invention. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of the invention. Conventional procedures for the selection and preparation of suitable prodrugs are described, for example, in Design of Prodrugs, ed. H. Bundgaard (Elsevier, 1985).

The term “protecting group” refers to a group of atoms that, when attached to a reactive functional group in a molecule, mask, reduce or prevent the reactivity of the functional group. Typically, a protecting group can be selectively removed as desired during the course of a synthesis. Examples of protecting groups can be found in Greene and Wuts, Protective Groups in Organic Chemistry, 3rd Ed. (John Wiley & Sons, New York 1999) and Harrison et al., Compendium of Synthetic Organic Methods, Vols. 1-8, 1971-1996 (John Wiley & Sons, New York). Representative nitrogen protecting groups include, but are not limited to, formyl, acetyl, trifluoroacetyl, benzyl, benzyloxycarbonyl CBZ), tert-butoxycarbonyl (Boc), trimethylsilyl (TMS), 2-trimethylsilyl-ethanesulfonyl (TES), trityl and substituted trityl groups, allyloxycarbonyl, 9-fluorenylmethyloxycarbonyl (FMOC), nitro-veratryloxycarbonyl (NVOC) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The term “substituted” refers to moieties having substituents replacing a hydrogen on one or more carbons of the backbone. The term “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. The term “substituted” include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen can have hydrogen substituents or any permissible substituents of organic compounds described herein that satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C1-6 alkyl, C3-6 cycloalkyl, halogen, carbonyl, cyano, or hydroxyl. In more preferred embodiments, the substituents on substituted alkyls are selected from fluoro, carbonyl, cyano, or hydroxyl. The substituents can themselves be substituted, if appropriate. Unless specifically stated as “unsubstituted,” references to chemical moieties are understood to include substituted variants. A reference to an “aryl” group or moiety implicitly includes both substituted and unsubstituted variants.

A “therapeutically effective amount” or a “therapeutically effective dose” of a drug or agent is an amount of a drug or an agent that, when administered to a subject will have the intended therapeutic effect. The precise effective amount needed for a subject will depend upon the subject's size, health and age, and the nature and extent of the condition being treated, such as MDS. One of ordinary skill in the pharmaceutical art can determine the effective amount for a given situation by routine experimentation. The effective amount of the compound will vary according to the weight, sex, age, and medical history of the subject. Other factors which influence the effective amount can include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to one skilled in the pharmaceutical art. See, Isselbacher et al. (1996) Harrison's Principles of Internal Medicine 13 ed., 1814-1882, incorporated herein by reference.

“Treating” a condition or patient refers to taking steps to obtain beneficial or desired results, including clinical results. “Treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

### Preferred Embodiments

The methods and techniques of the present disclosure are performed, unless otherwise indicated, according to conventional methods well known in the pharmaceutical art and as described in various general and more specific references that are cited and discussed throughout this specification. See, e.g., Principles of Neural Science (McGraw-Hill Medical, New York, 2000); Motulsky, Intuitive Biostatistics (Oxford University Press, Inc., 1995); Lodish et al., Molecular Cell Biology, 4th ed. (W. H. Freeman & Co., New York, 2000); Griffiths et al., Introduction to Genetic Analysis, 7th ed. (W. H. Freeman & Co., N.Y., 1999); and Gilbert et al., Developmental Biology, 6th ed. (Sinauer Associates, Inc., Sunderland, Mass., USA, 2000).

In a sixth embodiment, for a compound of formula I, NT is a guanine prodrug moiety with the following structure:

[chemical structure]

wherein R₅ is alkyl or aralkyl.

In a sixth embodiment, for a compound of formula I, NT is a thymine prodrug moiety with the following structure:

[chemical structure]

wherein R₅ is alkyl or aralkyl.

In a seventh embodiment, for a compound of formula I, NT is the moiety with the following structure:

[chemical structure]

In an eighth embodiment, fora compound of formula I, NT is a nucleobase, such as a natural nucleobase. In a ninth embodiment, for a compound of formula I, NT is adenine. In a tenth embodiment, for a compound of formula I, NT is guanine. In an eleventh embodiment, for a compound of formula I, NT is cytosine. In a twelfth embodiment, for a compound of formula I, NT is thymine.

In a thirteenth embodiment, for a compound of formula I, R¹ is a C₆-C₂₀ aryl or a 5-20 atom heteroaryl, such as phenyl, naphthyl, or 4-fluorophenyl. In a fourteenth embodiment, for a compound of formula I, R¹ is naphthyl. In a fifteenth embodiment, for a compound of formula I, R¹ is phenyl.

In a sixteenth embodiment, for a compound of formula I, R² and R^{2′}, each independently, is selected from hydrogen, C₁-C₆ alkyl, or C₇-C₁₆ aralkyl, or a natural amino acid side chain. In a seventeenth embodiment, for a compound of formula I, R² is selected from hydrogen or C₁-C₆ alkyl. In an eighteenth embodiment, for a compound of formula I, R² is hydrogen, methyl, isopropyl (i-propyl), or benzyl, most preferably methyl. In a nineteenth embodiment, for a compound of formula I, R² is a natural amino acid side chain. In a twentieth embodiment, for a compound of formula I, R^{2′} is methyl. In a twenty-first embodiment, for a compound of formula I, R^{2′} is hydrogen.

In a twenty-second embodiment, for a compound of formula I, the carbon to which R² is attached is in the S-configuration. In a twenty-third embodiment, for a compound of formula I, the carbon to which R² is attached is in the R-configuration. In a twenty-fourth embodiment, for a compound of formula I, the carbon to which R² is attached is in the D-configuration. In a twenty-fifth embodiment, for a compound of formula I, the carbon to which R² is attached is in the L-configuration (i.e., R² is disposed in the L-configuration).

In a twenty-sixth embodiment, for a compound of formula I, R³ is selected from C₁-C₆ alkyl or C₇-C₁₆ aralkyl, such as C₁-C₆ alkyl or C₇-C₁₁ aralkyl. In a twenty-seventh embodiment, for a compound of formula I, R³ is hydrogen, methyl, isopropyl, neopentyl, or benzyl.

In a twenty-eighth embodiment, for a compound of formula I, R⁴ is selected from hydrogen or C₁-C₆ alkyl, such as hydrogen or C₁-C₃ alkyl, e.g., methyl, ethyl, propyl, or isopropyl (i-propyl). In a twenty-ninth embodiment, for a compound of formula I, R⁴ is methyl. In a thirtieth embodiment, for a compound of formula I, R⁴ is hydrogen.

In a thirty-first embodiment, for a compound of formula I, R² and R⁴, together with the —C—N— moiety that separates them, form a 5-10-atom heterocycle, such as a 5-atom heterocycle. In a thirty-second embodiment, for a compound of formula I, R² and R⁴, together with the —C—N— moiety that separates them, form a pyrrolidine ring, e.g., as in proline.

In a thirty-third embodiment, for a compound of formula I, R₅ is selected from C₁-C₆ alkyl or C₇-C₁₆ aralkyl, such as C₁-C₆ alkyl or C₇-C₁₁ aralkyl e.g., methyl, ethyl, isopropyl (i-propyl), or benzyl. In a thirty-fourth embodiment, for a compound of formula I, R₅ is ethyl. In a thirty-fifth embodiment, for a compound of formula I, R₅ is methyl.

In a thirty-sixth embodiment, for a compound of formula I, R¹ is selected from phenyl, naphthyl, and halo-phenyl; and R⁴ is hydrogen. In a thirty-seventh embodiment, for a compound of formula I, R¹ is selected from phenyl, naphthyl, and fluoro-phenyl; and R⁴ is hydrogen. In a thirty-eighth embodiment, for a compound of formula I, R¹ is selected from phenyl, naphthyl, and 4-fluoro-phenyl; and R⁴ is hydrogen.

In a thirty-ninth embodiment, for a compound of formula I, R¹ is selected from phenyl, naphthyl, and halo-phenyl; R⁴ is hydrogen; R² is selected from alkyl and H; and R^{2′}, is selected from alkyl and H. In a fortieth embodiment, for a compound of formula I, R¹ is selected from phenyl, naphthyl, and halo-phenyl; R⁴ is hydrogen; R² is selected from alkyl and hydrogen; R^{2′} is selected from alkyl and H; and R² and R^{2′} are independently selected from alkyl and H. In a forty-first embodiment, for a compound of formula I, R¹ is selected from phenyl, naphthyl, and halo-phenyl; R⁴ is hydrogen; R² is hydrogen; R^{2′} is methyl.

In a forty-second embodiment, for a compound of formula I, R¹ is selected from phenyl, naphthyl, and halo-phenyl; R⁴ is hydrogen; and R³ is selected from alkyl, branched alkyl, and aralkyl. In a forty-third embodiment, for a compound of formula I, R¹ is selected from phenyl, naphthyl, and 4-fluoro-phenyl; R⁴ is hydrogen; and R³ is selected from methyl, isopropyl (i-propyl), and benzyl. In a forty-fourth embodiment, for a compound of formula I, R¹ is selected from phenyl, naphthyl, and halo-phenyl; R⁴ is hydrogen; R² is selected from alkyl and hydrogen; R^{2′} is selected from alkyl and hydrogen; R² and R^{2′} are independently selected from alkyl and hydrogen; and R³ is selected from methyl, isopropyl (i-propyl), and benzyl.

In a forty-fifth embodiment, for a compound of formula I, R¹ is selected from phenyl, naphthyl, and halo-phenyl; R⁴ is hydrogen; and NT is selected from adenine, guanine, cytosine, and thymine. In a forty-sixth embodiment, for a compound of formula I, R¹ is selected from phenyl, naphthyl, and 4-fluoro-phenyl; R⁴ is hydrogen; and NT is selected from adenine, guanine, cytosine, and thymine. In a forty-seventh embodiment, for a compound of formula I, R¹ is selected from phenyl, naphthyl, and halo-phenyl; R⁴ is hydrogen; R² is selected from alkyl and hydrogen; R^{2′} is selected from alkyl and hydrogen; R² and R^{2′} are independently selected from alkyl and hydrogen; and NT is selected from adenine, guanine, cytosine, and thymine. In a forty-eighth embodiment, for a compound of formula I, R¹ is selected from phenyl, naphthyl, and halo-phenyl; R⁴ is hydrogen; R² is selected from alkyl and hydrogen; R^{2′} is selected from alkyl and hydrogen; R² and R^{2′} are independently selected from alkyl and hydrogen; R³ is selected from methyl, isopropyl (i-propyl), and benzyl; and NT is selected from adenine, guanine, cytosine, and thymine.

In a forty-ninth embodiment, for a compound of formula I, R¹ is selected from phenyl, naphthyl, and halo-phenyl; R⁴ is hydrogen; and NT is selected from:

[chemical structure]

wherein R¹¹ is amino or hydrogen; and R¹² is alkyl or hydrogen.

In a fiftieth embodiment, for a compound of formula I, R¹ is selected from phenyl, naphthyl, and 4-fluoro-phenyl; R⁴ is hydrogen; and NT is selected from:

[chemical structure]

wherein R¹¹ is amino or hydrogen; and R¹² is alkyl or hydrogen.

In a fifty-first embodiment, for a compound of formula I, R¹ is selected from phenyl, naphthyl, and 4-fluoro-phenyl; R⁴ is hydrogen; R² is selected from alkyl and hydrogen; R^{2′} is selected from alkyl and H; R² and R^{2′} are independently selected from alkyl and H; and NT is selected from:

[chemical structure]

wherein R¹¹ is amino or hydrogen; and R¹² is alkyl or hydrogen.

In a fifty-second embodiment, for a compound of formula I, R¹ is selected from phenyl, naphthyl, and halo-phenyl; R⁴ is hydrogen; and the nucleobase prodrug moiety is selected from:

[chemical structure]

wherein R5 is selected from alkyl and aralkyl.

In a fifty-third embodiment, for a compound of formula I, R¹ is selected from phenyl, naphthyl, and halo-phenyl; R⁴ is hydrogen; NT is selected from adenine, guanine, cytosine, and thymine, and the nucleobase prodrug moiety is selected from:

[chemical structure]

wherein R5 is selected from alkyl and aralkyl.

In a fifty-third embodiment, for a compound of formula I, R¹ is selected from phenyl, naphthyl, and 4-fluoro-phenyl; R⁴ is hydrogen; NT is selected from adenine, guanine, cytosine, and thymine, and the nucleobase prodrug moiety is selected from:

[chemical structure]

wherein R5 is selected from alkyl and aralkyl.

In a fifty-fourth embodiment, the invention provides compounds having the structure of formula Ia:

[chemical structure]

and pharmaceutically acceptable salts and prodrugs thereof, wherein:
R¹ is aryl or heteroaryl;
R² is hydrogen, alkyl or aralkyl;
R³ is alkyl or aralkyl;
R⁴ is hydrogen or alkyl; and
NT is adenine, guanine, cytosine, or thymine.

In a fifty-fifth embodiment, for a compound of formula Ia, R¹ is phenyl, naphthyl, or 4-fluorophenyl; R² is methyl and the carbon to which R² is attached is in the L-configuration; R³ is methyl, benzyl, or isopropyl (i-propyl); or R⁴ is hydrogen. In a fifty-sixth embodiment, for a compound of formula Ia, R¹ is phenyl, naphthyl, or 4-fluorophenyl; R² is methyl and the carbon to which R² is attached is in the L-configuration; R³ is methyl, benzyl, or isopropyl (i-propyl); and R⁴ is hydrogen. In a fifty-seventh embodiment, fora compound of formula Ia, R¹ is naphthyl. In some preferred embodiments, R¹ is phenyl.

The invention provides compounds having the chemical structures depicted in Table 1, and pharmaceutically acceptable salts and prodrugs thereof.

TABLE 1

| A | G | T | C |
|---|---|---|---|
| [chemical structure] | [chemical structure] | [chemical structure] | [chemical structure] |
| [chemical structure] | [chemical structure] | [chemical structure] | [chemical structure] |
| [chemical structure] | [chemical structure] | [chemical structure] | [chemical structure] |
| [chemical structure] | [chemical structure] | [chemical structure] | [chemical structure] |
| [chemical structure] | [chemical structure] | [chemical structure] | [chemical structure] |
| [chemical structure] | [chemical structure] | [chemical structure] | [chemical structure] |
| [chemical structure] | [chemical structure] | [chemical structure] | [chemical structure] |
| [chemical structure] | [chemical structure] | [chemical structure] | [chemical structure] |
| [chemical structure] | [chemical structure] | [chemical structure] | [chemical structure] |

In a fifty-eighth embodiment, the compound is Compound 1017:

[chemical structure]

or a pharmaceutically acceptable salt or prodrug thereof.

In a fifty-ninth embodiment, the compound is Compound 15:

[chemical structure]

In a sixtieth embodiment, for a compound of formula I, R¹ is selected from phenyl. naphthyl, and 4-fluorophenyl; R² is methyl; R³ is selected from methyl, isopropyl, and benzyl; R⁴ is hydrogen; and NT is adenine, guanine, cytosine, thymine.

In a sixty-first embodiment, for a compound of formula I, R¹ is selected from phenyl, naphthyl, and 4-fluorophenyl; R² is methyl; R³ is selected from methyl, isopropyl, and benzyl; R⁴ is hydrogen; and NT is selected from:

[chemical structure]

wherein R¹¹ is amino or hydrogen; and R¹² is alkyl or hydrogen.

In a sixty-second embodiment, for a compound of formula II, R¹ is hydrogen.

In a sixty-third embodiment, for a compound of formula II, R^{2a} is alkyl or aralkyl. In a sixty-fourth embodiment, for a compound of formula II, R^{2a} is methyl, isopropyl (i-propyl), or benzyl. In a sixty-fifth embodiment, for a compound of formula II, R^{2a} is methyl.

In a sixty-sixth embodiment, fora compound of formula II, R^{2b} is hydrogen, alkyl or aralkyl. In a sixty-seventh embodiment, for a compound of formula II, R^{2b} is hydrogen. In a sixty-eighth embodiment, for a compound of formula II, R^{2b} is H and R^{2a} is alkyl or aralkyl. Alternatively, R^{2b} is hydrogen and R^{2a} is methyl, isopropyl (i-propyl), or benzyl. In a sixty-ninth embodiment, fora compound of formula II, R^{2b} is hydrogen and R^{2a} is methyl.

In a seventieth embodiment, for a compound of formula II, R³ is methyl, neopentyl or isopropyl (i-propyl). In a seventy-first embodiment, for a compound of formula II, R³ is isopropyl (i-propyl).

In a seventy-second embodiment, for a compound of formula II, NT is a nucleobase. In a seventy-third embodiment, for a compound of formula II, NT is adenine, guanine, cytosine, or thymine. In certain embodiments, NT is adenine. In a seventy-fourth embodiment, for a compound of formula II, NT is guanine. In a seventy-fifth embodiment, for a compound of formula II, NT is cytosine. In a seventy-sixth embodiment, for a compound of formula II, NT is thymine.

In a seventy-seventh embodiment, for a compound of formula II, NT is a nucleobase prodrug moiety. In a seventy-eighth embodiment, for a compound of formula II, the nucleobase prodrug moiety is

[chemical structure]

where R5 is alkyl or aralkyl. In a seventy-ninth embodiment, for a compound of formula II, R5 is methyl, ethyl, isopropyl (i-propyl), or benzyl. In an eightieth embodiment, for a compound of formula II, R5 is methyl.

In an eighty-first embodiment, the compound is 2005:

[chemical structure]

a pharmaceutically acceptable salt thereof.

In an eighty-first embodiment, the compound is 2005:

[chemical structure]

### Characteristics of the Preferred Embodiments

A Log of solubility, Log S or log S is used in the pharmaceutical art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

The compounds of the invention can be prodrugs of dNMPs, and can be used to treat MDSs, or for any other purpose for which dNMP prodrugs, or dNMPs themselves, are useful in the treatment of disease. The prodrug compounds of the invention are expected to have desirable physicochemical properties, given their calculated log P (octanol-water partition), log S (solubility in water), and TPSA (total polar surface area) values all indicate that they will efficiently cross cell membranes and be readily solvated in biological fluids. Those calculated values are given in Table 2.

TABLE 2

| Compound | NT | R¹ | R² | R³ | R⁴ | log P | log S | TPSA |
|---|---|---|---|---|---|---|---|---|
| 1 | A | Ph | L-Me | Me | H | 0.34 | −3.73 | 173 |
| 2 | A | Ph | L-Me | Bn | H | 2.12 | −5.49 | 173 |
| 3 | A | Ph | L-Me | iPr | H | 1.14 | −4.38 | 173 |
| 4 | A | Np | L-Me | Me | H | 1.56 | −5.60 | 173 |
| 5 | A | Np | L-Me | Bn | H | 3.34 | −7.37 | 173 |
| 6 | A | Np | L-Me | iPr | H | 2.36 | −6.26 | 173 |
| 7 | A | 4-FPh | L-Me | Me | H | 0.49 | −4.02 | 173 |
| 8 | A | 4-FPh | L-Me | Bn | H | 2.28 | −5.79 | 173 |
| 9 | A | 4-FPh | L-Me | iPr | H | 1.29 | −4.67 | 173 |
| 10 | G | Ph | L-Me | Me | H | -0.23 | −3.43 | 189 |
| 11 | G | Ph | L-Me | Bn | H | 1.55 | −5.20 | 189 |
| 12 | G | Ph | L-Me | iPr | H | 0.57 | −4.08 | 189 |
| 13 | G | Np | L-Me | Me | H | 0.99 | −5.31 | 189 |
| 14 | G | Np | L-Me | Bn | H | 2.77 | −7.07 | 189 |
| 15 | G | Np | L-Me | iPr | H | 1.79 | −5.96 | 189 |
| 16 | G | 4-FPh | L-Me | Me | H | -0.08 | −3.72 | 189 |
| 17 | G | 4-FPh | L-Me | Bn | H | 1.71 | −5.49 | 189 |
| 18 | G | 4-FPh | L-Me | iPr | H | 0.72 | −4.38 | 189 |
| 19 | T | Ph | L-Me | Me | H | -0.15 | −2.65 | 153 |
| 20 | T | Ph | L-Me | Bn | H | 1.64 | −4.42 | 153 |
| 21 | T | Ph | L-Me | iPr | H | 0.65 | −3.31 | 153 |
| 22 | T | Np | L-Me | Me | H | 1.07 | −4.53 | 153 |
| 23 | T | Np | L-Me | Bn | H | 2.86 | −6.30 | 153 |
| 24 | T | Np | L-Me | iPr | H | 1.87 | −5.18 | 153 |
| 25 | T | 4-FPh | L-Me | Me | H | 0.00 | −2.95 | 153 |
| 26 | T | 4-FPh | L-Me | Bn | H | 1.79 | −4.71 | 153 |
| 27 | T | 4-FPh | L-Me | iPr | H | 0.81 | −3.60 | 153 |
| 28 | C | Ph | L-Me | Me | H | 0.13 | −2.83 | 162 |
| 29 | C | Ph | L-Me | Bn | H | 1.91 | −4.60 | 162 |
| 30 | C | Ph | L-Me | iPr | H | 0.93 | −3.49 | 162 |
| 31 | C | Np | L-Me | Me | H | 1.35 | −4.71 | 162 |
| 32 | C | Np | L-Me | Bn | H | 3.13 | −6.48 | 162 |
| 33 | C | Np | L-Me | iPr | H | 2.15 | −5.37 | 162 |
| 34 | C | 4-FPh | L-Me | Me | H | 0.28 | −3.13 | 162 |
| 35 | C | 4-FPh | L-Me | Bn | H | 2.07 | −4.90 | 162 |
| 36 | C | 4-FPh | L-Me | iPr | H | 1.08 | −3.78 | 162 |

The compounds of the invention can be prodrugs of the compounds of Table I, e.g., wherein a hydroxyl in the parent compound is presented as an ester or a carbonate, or carboxylic acid present in the parent compound is presented as an ester. In certain such embodiments, the prodrug is metabolized to the active parent compound in vivo (e.g., the ester is hydrolyzed to the corresponding hydroxyl, or carboxylic acid).

The compounds of the invention can be racemic. In certain embodiments, compounds of the invention can be enriched in one enantiomer. For example, a compound of the invention can have greater than 30% ee, 40% ee, 50% ee, 60% ee, 70% ee, 80% ee, 90% ee, or even 95% or greater ee. In certain embodiments, compounds of the invention can have more than one stereocenter. In certain such embodiments, compounds of the invention can be enriched in one or more diastereomers. For example, a compound of the invention can have greater than 30% de, 40% de, 50% de, 60% de, 70% de, 80% de, 90% de, or even 95% or greater de.

Many of the compounds useful in the methods and compositions of this invention have at least one stereogenic center in their structure. This stereogenic center can be present in an R or an S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups can exist as Z (zusammen) or E (entgegen) isomers. In each instance, the disclosure includes both mixture and separate individual isomers.

Some of the compounds can also exist in tautomeric forms. Such forms, although not explicitly indicated in the formulae described herein, are included within the scope of the disclosure.

### Medical Use

The invention relates to the use of a compound of formula I, formula Ia, formula II, or a compound selected from Table I, or a pharmaceutically acceptable salt thereof in the treatment of diseases. The use involves the administering the compound of the invention to a patient in need thereof.

The therapeutic preparation can be enriched to provide predominantly one enantiomer of a compound (e.g., of a compound selected from Table I). An enantiomerically enriched mixture can comprise, for example, at least 60 mol percent of one enantiomer, or more preferably at least 75, 90, 95, or even 99 mol percent. The compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

The therapeutic preparation can be enriched to provide predominantly one diastereomer of a compound (e.g., of a compound selected from Table 1). A diastereomerically enriched mixture can comprise, for example, at least 60 mol percent of one diastereomer, or more preferably at least 75, 90, 95, or even 99 mol percent.

The invention provides a pharmaceutical preparation suitable for use in a human patient, comprising any of the compounds shown above (e.g., a compound of the invention, such as a compound of Formula I or (Ia) or a compound selected from Table 1), and one or more pharmaceutically acceptable excipients. The pharmaceutical preparations can be for use in treating or preventing a condition or disease as described herein.

Compounds of any of the above structures can be used in the manufacture of medicaments for the treatment of any diseases or conditions disclosed herein.

### Use of Deoxynucleotide Prodrugs

The invention provides a method of treating a patient suffering from an MDS, by administering to the patient a therapeutically effective amount of a compound of Formula I, Formula Ia, Formula II, such as compound 2005. The MDS to be treated is selected from DGUOK deficiency, TK2 deficiency, MNGIE, POLG deficiency, Alpers-Huttenlocher syndrome, SANDO syndrome, MIRAS, MPV17-related hepatocerebral myopathy, or RRM2B-related myopathy.

In an eighty-second embodiment, the MDS is an RRM2B-related myopathy. In some embodiments, the MDS is linked to a mutation in TK2, DGUOK, POLG, MPV17, RRM2B, SUCLA2, SUCLG1, TYMP, C10orf2, or SAMHD1. In an eighty-third embodiment, the MDS has unknown pathophysiology.

In an eighty-fourth embodiment, the dAMP and dGMP prodrugs of the invention, i.e., the compounds of formula I, formula Ia, or formula II, such as compound 2005, wherein NT is adenine or guanine or an adenine or guanine prodrug moiety, can be used to treat DGUOK deficiency. In an eighty-fifth embodiment, NT is adenine or guanine. In other such embodiments, NT is an adenine or guanine prodrug moiety.

In an eighty-sixth embodiment, the dCTP and dTTP prodrugs of the invention, i.e., the compounds of formula I, formula Ia, or formula II, wherein NT is cytosine or thymine or a cytosine or thymine prodrug moiety, can be used to treat TK2 deficiency. In an eighty-seventh embodiment, NT is thymine or cytosine. In an eighty-eighth embodiment, NT is a thymine or cytosine prodrug moiety.

In an eighty-ninth embodiment, the dCTP prodrugs of the invention, i.e., the compounds wherein NT is cytosine or a cytosine prodrug moiety, can be used to treat MNGIE. In a ninetieth embodiment, NT is cytosine. In a ninety-first embodiment, NT is a cytosine prodrug moiety.

In a ninety-second embodiment, the dAMP, dGMP, dCTP, and dTTP prodrugs of the invention, i.e., the compounds wherein NT is adenine, guanine, cytosine, or thymine or an adenine, guanine, cytosine, or thymine prodrug moiety, can be used to treat POLG deficiency. In a ninety-third embodiment, NT is adenine or guanine or an adenine or guanine prodrug moiety. In a ninety-fourth embodiment, NT is adenine or guanine. In a ninety-fifth embodiment, NT is an adenine or guanine prodrug moiety.

In a ninety-sixth embodiment, the dAMP and dGMP prodrugs of the invention, i.e., the compounds wherein NT is adenine or guanine or an adenine or guanine prodrug moiety, can be used to treat MPV17. In a ninety-seventh embodiment, NT is adenine or guanine. In other such embodiments, NT is an adenine or guanine prodrug moiety.

In a ninety-eighth embodiment, the dAMP, dGMP, dCTP, and dTTP prodrugs of the invention, i.e., the compounds wherein NT is adenine, guanine, cytosine, or thymine or an adenine, guanine, cytosine, or thymine prodrug moiety, can be used to treat a mitochondrial DNA depletion syndrome that is linked to a mutation in SAMDH1. In a ninety-ninth embodiment, NT is adenine, guanine, thymine or cytosine. In a one hundredth embodiment, NT is an adenine, guanine, thymine or cytosine prodrug moiety.

In a one hundred and first embodiment, the dAMP, dGMP, dCTP, and dTTP prodrugs of the invention, i.e., the compounds of Formula I wherein NT is adenine, guanine, cytosine, or thymine or an adenine, guanine, cytosine, or thymine prodrug moiety, can be used to treat a mitochondrial DNA depletion syndrome that is linked to a mutation in RR2 MB. In a one hundred and second embodiment, NT is adenine, guanine, thymine or cytosine. In a one hundred and third embodiment, NT is an adenine, guanine, thymine or cytosine prodrug moiety.

### Pharmaceutical Compositions

The compositions and methods of the invention can be used to treat an individual in need thereof. The individual can be a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and excipients can be chosen to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound can also be formulated for inhalation. In a one hundred and fourth embodiment, a compound can be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations can conveniently be presented in unit dosage form and can be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Formulations of the invention suitable for oral administration can be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. Compositions or compounds can also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, can optionally be scored or prepared with coatings and shells, such as enteric coatings and other coatings well known in the pharmaceutical-formulating art. They can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions that can be dissolved in sterile water, or some other sterile injectable medium immediately before use. These compositions can also optionally contain opacifying agents and can be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms can contain inert diluents commonly used in the art, such as, for example, water or other solvents, cyclodextrins and derivatives thereof, solubilizing agents and emulsifiers, such as ethyl alcohol, isopropyl (i-propyl) alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound can be mixed under sterile conditions with a pharmaceutically acceptable carrier, and with any preservatives, buffers, or propellants that can be required.

The ointments, pastes, creams and gels can contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Pharmaceutical compositions suitable for parenteral administration comprise one or more active compounds in combination with one or more pharmaceutically-acceptable sterile isotonic aqueous or nonaqueous solutions, dispersions, suspensions or emulsions, or sterile powders which can be reconstituted into sterile injectable solutions or dispersions just prior to use, which can contain antioxidants, buffers, bacteriostats, solutes which render the formulation isotonic with the blood of the intended recipient or suspending or thickening agents.

Examples of suitable aqueous and nonaqueous carriers that can be used in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents that delay absorption such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution, which, in turn, can depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide.

Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction can also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions can be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved.

A suitable daily dose of an active compound used in the compositions and methods of the invention can be that amount of the compound that is the lowest dose effective to produce a therapeutic effect. Such an effective dose will generally depend upon the factors described above.

The effective daily dose of the active compound can be administered as one, two, three, four, five, six or more sub-doses administered separately at appropriate intervals throughout the day, optionally, in unit dosage forms. In a one hundred and fifth embodiment, the active compound can be administered two or three times daily. In a one hundred and sixth embodiment, the active compound will be administered once daily.

In a one hundred and seventh embodiment, compounds of the invention can be used alone or conjointly administered with another type of therapeutic agent.

The disclosure includes the use of pharmaceutically acceptable salts of compounds of the invention in the compositions and methods of the invention. In a one hundred and eighth embodiment, contemplated salts of the invention include, but are not limited to, alkyl, dialkyl, trialkyl or tetra-alkyl ammonium salts. In a one hundred and ninth embodiment, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In a one hundred and tenth embodiment, contemplated salts of the invention include, but are not limited to, Na, Ca, K, Mg, Zn or other metal salts. In a one hundred and eleventh embodiment, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d glucoheptonic acid, d gluconic acid, d glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The following Example is provided to illustrate the invention, and should not be considered to limit its scope in any way.

### EXAMPLE

### Example 1: Synthetic Protocols (Method A)

[chemical structure]

### General Procedure for the Preparation of Compound 12 isopropyl ((((2R,3S,5R)-5-(2-amino-6-oxo-1,6-dihydro-9H-purin-9-yl)-3-hydroxytetrahydrofuran-2-yl)methoxy)(phenoxy)phosphoryl)-L-alaninate

To a solution of compound 1001 (25.0 g, 118.5 mmol, 1.0 eq) in dichloromethane (250 mL) at −78° C. was added a solution of 4-nitrophenol (16.5 g, 118.5 mmol, 1.0 eq) in dichloromethane (250 mL) and TEA (18 mL, 130.3 mmol, 1.1 eq). The reaction mixture was warmed to room temperature, stirred for 1 h, and cooled to 0° C. A solution of compound 1003 (19.9 g, 118.5 mmol, 1.0 eq) and triethylamine (34.5 mL, 248.9 mmol, 2.1 eq) in dichloromethane (250 mL) was added. The mixture was warmed to room temperature, stirred for 2 h, and quenched with water (500 mL). The organic layer was separated, dried over sodium sulfate, filtered, and concentrated under reduced pressure. The crude product was purified by flash column chromatography on silica gel (Et₂O/EtOAc=2/1) to give compound 1004 (25.0 g, 52%) as colorless oil. LC-MS: 409.2 [M+H]⁺, expected 409.11, ¹H NMR (400 MHz, CDCl₃) (δ, ppm) 8.18 (d, J=8.7 Hz, 2H), 7.34 (ddd, J=15.9, 12.9, 5.1 Hz, 4H), 7.27-7.08 (m, 3H), 4.98 (m, 1H), 4.36-4.16 (m, 1H), 1.35 (d, J=7.0 Hz, 3H), 1.24-1.15 (m, 6H).

To a solution of compound 1005 (2.6 g, 9.8 mmol, 1.0 eq) in THF (7.5 mL) and NMP (30 mL) at 0° C. was added 1.0 M t-BuMgCl (14.8 mL, 14.7 mmol, 1.5 eq). The mixture was stirred at 0° C. for 0.5 h and a solution of compound 1004 (3.0 g, 7.35 mmol, 0.75 eq) in THF (10 mL) was added. The mixture was warmed to room temperature and stirred overnight. A saturated aqueous solution of NH₄Cl (30 mL) was added and the organic phase was extracted with ethyl acetate (2×50 mL). The combined organic layer was dried with sodium sulfate, filtered, and concentrated. The crude product was purified by column chromatography on silica gel twice (DCM-DCM/MeOH=15/1) to afford compound 12 (600 mg, 17%, >95% purity) as a white solid.

The following compounds were prepared according to the general procedure described in Method A via displacement of the 4-nitrophenol leaving group with the appropriate deoxynucleoside base.

| | | Observed | |
|---|---|---|---|
| Comp. | | MW | ¹H NMR |
| No. | IUPAC Name | (Expected) | (400 MHz) |
| 12 | isopropyl ((((2R,3S,5R)-5-(2- | 537.2 | CD₃OD (δ, ppm) 7.97 (s, 1H), |
| (G) | amino-6-oxo-1,6-dihydro- | (537.3) | 7.35-7.25 (m, 2H), 7.17 (td, J = |
| | 9H-purin-9-yl)-3- | | 15.9, 7.5 Hz, 3H), 6.28 (dd, J = |
| | hydroxytetrahydrofuran-2- | | 13.6, 6.5 Hz, 1H), 4.59 (m, 1H), |
| | yl)methoxy)(phenoxy)phosphoryl)- | | 4.37 (m, 2H), 4.28 (m, 1H), 4.16 |
| | L-alaninate | | (m, 1H), 3.86 (m, 1H), 2.66 (m, |
| | | | 1H), 2.40 (m, 1H), 1.28 (t, J = 6.4 |
| | | | Hz, 3H), 1.23-1.14 (m, 6H) |
| 18 | isopropyl ((((2R,3S,5R)-5-(2- | 555.2 | CD₃OD (δ, ppm) 7.92 (m, 1H), |
| (G) | amino-6-oxo-1,6-dihydro- | (555.2) | 7.30-7.15 (m, 2H), 7.05 (m, 2H), |
| | 9H-purin-9-yl)-3- | | 6.28 (m, 1H), 4.59 (m, 1H), 4.37 |
| | hydroxytetrahydrofuran-2- | | (m, 2H), 4.28 (m, 1H), 4.16 (m, |
| | yl)methoxy)(4- | | 1H), 3.84 (m, 1H), 2.70 (m, 1H), |
| | fluorophenoxy)phosphoryl)- | | 2.38 (m, 1H), 1.24 (t, J = 6.4 Hz, |
| | L-alaninate | | 3H), 1.23-1.14 (m, 6H) |
| 15 | isopropyl ((((2R,3S,5R)-5-(2- | 587.2 | CD₃OD (δ, ppm) 8.10 (m, 1H), |
| (G) | amino-6-oxo-1,6-dihydro- | (587.3) | 7.88 (m, 1H), 7.68 (m, 1H), 7.50 |
| | 9H-purin-9-yl)-3- | | (m, 3H), 7.38 (m, 2H), 6.24 (m, |
| | hydroxytetrahydrofuran-2- | | 1H), 4.55 (m, 1H), 4.40 (m, 2H), |
| | yl)methoxy)(naphthalen-1- | | 4.36 (m, 1H), 4.18 (m, 1H), 3.98 |
| | yloxy)phosphoryl)-L-alaninate | | (m, 1H), 2.45 (m, 1H), 2.30 (m, |
| | | | 1H), 1.28 (m, 3H), 1.20 ? 1.14 (m, |
| | | | 6H) |
| 21 | isopropyl ((((2R,3S,5R)-3- | 512.2 | CDCl₃ (δ, ppm) 8.85 (brs, 1H), |
| (T) | hydroxy-5-(5-methyl-2,4-dioxo- | (512.3) | 7.39-7.28 (m, 3H), 7.24-7.11 |
| | 3,4-dihydropyrimidin- | | (m, 3H), 6.25 (m, 1H), 5.08-4.90 |
| | 1(2H)-yl)tetrahydrofuran-2- | | (m, 1H), 4.48 (m, 1H), 4.32 (m, |
| | yl)methoxy)(phenoxy)phosphoryl) | | 1H), 4.10-3.90 (m, 3H), 3.80- |
| | -L-alaninate | | 3.60 (m, 1H), 2.42-2.29 (m, 1H), |
| | | | 2.12 (m, 1H), 1.89 (d, J = 3.6 Hz, |
| | | | 3H), 1.77 (m, 1H), 1.35 (m, 3H), |
| | | | 1.22 (m, 6H) |
| 27 | isopropyl ((4- | 530.2 | CDCl₃ (δ, ppm) 9.49 (s, 1H), 7.35 |
| (T) | fluorophenoxy)(((2R,3S,5R)- | (530.3) | (s, 1H), 7.16 (m, 2H), 6.99 (m, |
| | 3-hydroxy-5-(5-methyl-2,4- | | 2H), 6.27 (m, 1H), 5.05-4.84 (m, |
| | dioxo-3,4-dihydropyrimidin- | | 1H), 4.47 (m, 1H), 4.31 (m, 2H), |
| | 1(2H)-yl)tetrahydrofuran-2- | | 4.21 (m, 1H), 4.08 (m, 1H), 4.00- |
| | yl)methoxy)phosphoryl)-L- | | 3.87 (m, 1H), 2.36 (s, 1H), 2.15 |
| | alaninate | | (m, 2H), 1.85 (d, J = 19.8 Hz, 3H), |
| | | | 1.34 (t, J =7.6 Hz, 3H), 1.20 (m, |
| | | | 6H) |
| 24 | isopropyl ((((2R,3S,5R)-3- | 562.2 | CDCl₃ (δ, ppm) 8.85 (m, 1H), 8.05 |
| (T) | hydroxy-5-(5-methyl-2,4- | (562.2) | (m, 1H), 7.83 (m, 1H), 7.65 (m, |
| | dioxo-3,4-dihydropyrimidin- | | 1H), 7.57-7.44 (m, 3H), 7.44- |
| | 1(2H)-yl)tetrahydrofuran-2- | | 7.33 (m, 2H), 7.31 (m, 1H), 6.24 |
| | yl)methoxy)(naphthalen-1- | | (m, 1H), 5.01-4.87 (m, 1H), 4.39 |
| | yloxy)phosphoryl)-L-alaninate | | (m, 3H), 4.05 (m, 4H), 2.28 (m, |
| | | | 1H), 1.98 (m, 1H), 1.78 (d, J = |
| | | | 18.6 Hz, 3H), 1.32 (dd, J = 10.9, |
| | | | 6.8 Hz, 3H), 1.20 (d, J = 6.2 Hz, |
| | | | 3H), 1.16 (dd, J = 9.4, 6.3 Hz, 3H) |
| 30 | isopropyl ((((2R,3S,5R)-5-(4- | 497.2 | CD₃OD (δ, ppm) 7.82 (m, 1H), |
| (C) | amino-2-oxopyrimidin-1(2H)- | (497.5) | 7.38 (m, 2H), 7.22 (m, 3H), 6.24 |
| | yl)-3- | | (m, 1H), 5.84 (m, 1H), 4.38 (m, |
| | hydroxytetrahydrofuran-2- | | 2H), 4.30 (m, 2H), 4.14 (m, 1H), |
| | yl)methoxy)(phenoxy)phosphoryl)- | | 3.90 (m, 1H), 2.32 (m, 1H), 1.98 |
| | L-alaninate | | (m, 1H), 1.34 (m, 3H), 1.20 (m, |
| | | | 6H) |
| 36 | isopropyl ((((2R,3S,5R)-5-(4- | 515.2 | CD₃OD (δ, ppm) 7.82 (m, 1H), |
| (C) | amino-2-oxopyrimidin-1(2H)- | (515.4) | 7.28 (m, 2H), 7.08 (m, 3H), 6.28 |
| | yl)-3- | | (m, 1H), 5.94 (m, 1H), 4.38 (m, |
| | hydroxytetrahydrofuran-2- | | 2H), 4.30 (m, 2H), 4.10 (m, 1H), |
| | yl)methoxy)(4- | | 3.90 (m, 1H), 2.36 (m, 1H), 2.04 |
| | fluorophenoxy)phosphoryl)- | | (m, 1H), 1.34 (m, 3H), 1.24 (m, |
| | L-alaninate | | 6H) |
| 33 | isopropyl ((((2R,3S,5R)-5-(4- | 547.2 | CD₃OD (δ, ppm) 8.18 (m, 1H), |
| (C) | amino-2-oxopyrimidin-1(2H)- | (547.3) | 7.88 (m, 1H), 7.70 (m, 1H),7.56 |
| | yl)-3- | | (m, 3H), 7.54 (m, 1H), 7.44 (m, |
| | hydroxytetrahydrofuran-2- | | 1H), 6.20 (m, 1H), 5.76 (m, 1H), |
| | yl)methoxy)(naphthalen-1- | | 4.38 (m, 1H), 4.30 (m, 3H), 4.10 |
| | yloxy)phosphoryl)-L-alaninate | | (m, 1H), 3.98 (m, 1H), 2.18 (m, |
| | | | 1H), 1.64 (m, 1H), 1.34 (m, 3H), |
| | | | 1.22 (m, 6H) |

### Example 2: Synthetic Protocols (Method B)

[chemical structure]

### General Procedure for the Preparation of Compound 1017 isopropyl ((((2R, 3S, 5R)-5-(2-amino-6-methoxy-9H-purin-9-yl)-3-hydroxytetrahydrofuran-2-yl)methoxy)(naphthalen-1-yloxy)phosphoryl)-L-alaninate

To a solution of compound 1005 (3.0 g, 11.2 mmol, 1.0 eq) in MeOH (200 mL) at −20° C. was added excess CH₂N₂ etherate and stirred for 4 h. The reaction was monitored by LCMS. The resulting mixture was concentrated, triturated with MeOH, and filtered. The filtrate was concentrated to afford crude compound 1015 (2.5 g, 79%) as white powder, which was used for next step without further purification. To a solution of compound 1016 (2.0 g, 4.37 mmol, 1.0 eq) in THF (6 mL) and NMP (25 mL) at 0° C. was added 1.0 M t-BuMgCl (6.55 mL, 6.55 mmol, 1.5 eq). The mixture was stirred at 0° C. for 0.5 h and a solution of compound 1015 (2.5 g, 8.9 mmol, 2.04 eq) in THF (8 mL) was added. The mixture was warmed to room temperature and stirred for 16 h. A saturated aqueous solution of NH₄Cl (25 mL) was added and the organic phase was extracted with ethyl acetate (2×40 mL). The combined organic layer was dried over sodium sulfate, filtered, and concentrated. The crude product was dissolved in MeOH and purified by prep-HPLC to afford compound 1017 (158 mg, 6%, >95% purity) as white solid. LCMS: m/z (ESI+) 601.3 [M+1]⁺, expected 601.2, ¹H NMR (400 MHz, DMSO-d6) δ 8.09 (t, J=6.4 Hz, 1H), 7.95-7.87 (m, 2H), 7.71 (t, J=7.2 Hz, 1H), 7.56-7.43 (m, 2H), 7.40-7.35 (m, 2H), 6.44 (d, J=2.4 Hz, 2H), 6.23-6.13 (m, 2H), 5.46 (t, J=4.8 Hz, 1H), 4.84-4.73 (m, 1H), 4.42-4.39 (m, 1H), 4.34-4.28 (m, 1H), 4.24-4.18 (m, 1H), 4.12-4.04 (m, 1H), 3.98 (s, 3H), 3.93-3.76 (m, 1H), 2.61-2.47 (m, 1H), 2.25-2.13 (m, 1H), 1.19 (d, J=7.2 Hz, 3H), 1.09-1.03 (m, 6H).

### Example 3: Synthetic Protocols (Method C)

[chemical structure]

### General Procedure for the Preparation of Compound 1023 neopentyl ((((2R,3S,5R)-5-(2-amino-6-oxo-1,6-dihydro-9H-purin-9-yl)-3-hydroxytetrahydrofuran-2-yl)methoxy)(naphthalen-1-yloxy)phosphoryl)-L-alaninate

To a mixture of compound 1018 (10 g, 52.8 mmol, 1.0 eq) and neopentyl alcohol (5.58 g, 63.4 mmol, 1.2 eq) in DCM (100 mL) at 0° C. under nitrogen atmosphere was added DMAP (0.64 g, 5.28 mmol, 0.1 eq) and EDCl.HCl (15.2 g, 79.3 mmol, 1.5 eq). The reaction mixture was warmed to room temperature and stirred for 16 h. The reaction was monitored by TLC. The mixture was extracted with ethyl acetate (3×100 mL). The organic phase was washed with brine, dried over Na₂SO₄ and concentrated under reduced pressure. The residue was purified by flash chromatography on silica (Et₂O/EtOAc=30:1) to give compound 1019 (12.5 g, 91%) as colorless oil.

To a solution of compound 1019 (6.16 g, 23.8 mmol, 1.0 eq) in HCl/EtOAc solution (2 M, 50 mL, 100 mmol) was stirred at room temperature for 1 h. The reaction was monitored by ¹H NMR. The mixture was concentrated under reduced pressure to give compound 1020 (4.42 g, 95%) as white powder.

A mixture of compound 1020 (1.0 g, 5.1 mmol, 1.0 eq), compound 1021 (3.7 g, 10.2 mmol, 2.0 eq) in DCM (10 mL) at 0° C. was added triethylamine (2.23 mL, 16.1 mmol, 3.15 eq). The reaction was monitored by TLC. Then the mixture was extracted with EtOAc (3×20 mL). The organic phase was washed with brine, dried over sodium sulfate and concentrated under reduced pressure. The residue was purified by flash chromatography on silica (Et₂O/EtOAc=50:1-30:1-1:1) to give compound 1022 (650 mg, 26%) as a white solid.

To a solution of compound 1022 (660 mg, 1.36 mmol, 1.0 eq) in THF (2.77 mL) and NMP (8.32 mL) at 0° C. was added 1.0 M t-BuMgCl (4.09 mL, 4.08 mmol, 3.0 eq). The mixture was stirred at 0° C. for 0.5 h and a solution of compound 1005 (726 mg, 2.72 mmol, 2.0 eq) in THF (2.77 mL) was added. The mixture was warmed to room temperature and stirred for 16 h. The reaction was monitored by LCMS. A saturated aqueous solution of NH₄Cl (5 mL) was added and the organic phase was extracted with EtOAc (2×10 mL). The combined organic layers were dried over Na₂SO₄, filtered, and concentrated. The crude product was purified by prep-HPLC to afford 1023 (100 mg, 12%, >95% purity) as a white powder. LCMS: m/z (ESI+) 615.4 [M+H]⁺, expected 615.2, ¹H NMR (400 MHz, DMSO-d6) δ 10.59 (d, J=2.8 Hz, 1H), 8.10 (t, J=9.6 Hz, 1H), 7.94-7.89 (m, 1H), 7.78-7.56 (m, 2H), 7.54-7.49 (m, 2H), 7.46-7.37 (m, 2H), 6.43 (d, J=4.0 Hz, 2H), 6.23-6.16 (m, 1H), 6.13-6.09 (m, 1H), 5.40 (t, J=4.4 Hz, 1H), 4.38-4.22 (m, 1H), 4.14-4.02 (m, 2H), 3.98-3.87 (m, 2H), 3.74-3.71 (m, 1H), 3.70-3.59 (m, 1H), 2.48-2.33 (m, 1H), 2.21-2.11 (m, 1H), 1.25-1.22 (m, 3H), 0.82 (s, 9H).

### Example 4: Synthetic Protocols (Method D)

[chemical structure]

### General Procedure for the Preparation of Compound 14 benzyl ((((2R,3S,5R)-5-(2-amino-6-oxo-1,6-dihydro-9H-purin-9-yl)-3-hydroxytetrahydrofuran-2-yl)methoxy)(naphthalen-1-yloxy)phosphoryl)-L-alaninate

A solution of compound 1024 (10.0 g, 46.5 mmol, 1.0 eq), compound 1021 (33.8 g, 93 mmol, 2.0 eq) and TEA (13.5 mL, 97.7 mmol, 2.1 eq) in DCM (120 mL) was stirred at 0° C. The mixture was warmed to room temperature and stirred for 2 h. The reaction was monitored by LCMS. The resulting mixture was quenched with water (250 mL). The organic layer was separated, dried over Na₂SO₄, filtered, and concentrated under reduced pressure. The crude product was purified by flash chromatography on silica (Et₂O/EtOAc=2/1) to give compound 1025 (14 g, 59%) as colorless oil.

To a solution of compound 1025 (5.0 g, 9.88 mmol, 1.0 eq) in THF (15 mL) and NMP (60 mL) at 0° C. was added 1.0 M t-BuMgCl (14.8 mL, 14.8 mmol, 1.5 eq). The mixture was stirred at 0° C. for 0.5 h and a solution of compound 7a (1.98 g, 7.41 mmol, 0.75 eq) in THF (8 mL) was added. The mixture was warmed to room temperature and stirred for 16 h. A saturated aqueous solution of NH₄Cl (60 mL) was added and the organic phase was extracted with ethyl acetate (2×100 mL). The combined organic layer was dried over Na₂SO₄, filtered, and concentrated. The crude product was purified by prep-HPLC to afford compound 14 (180 mg, 3%, >95% purity) as white solid. LCMS: m/z (ESI+) 635.3 [M+H]⁺, expected 635.2, ¹H NMR (400 MHz, DMSO-d6) δ 10.57 (s, 1H), 8.09 (d, J=7.6 Hz, 1H), 7.92 (d, J=7.2 Hz, 1H), 7.77 (s, 1H), 7.71 (d, J=7.6 Hz, 1H), 7.56-7.48 (m, 2H), 7.45-7.36 (m, 2H), 7.28 (s, 5H), 6.43 (s, 2H), 6.29-6.22 (m, 1H), 6.12 (t, J=6.0 Hz, 1H), 5.40 (d, J=4.0 Hz, 1H), 5.02 (dd, J=12.4, 12.4 Hz, 2H), 4.38-4.32 (m, 1H), 4.26-4.20 (m, 1H), 4.12-4.05 (m, 1H), 4.01-3.93 (m, 2H), 2.48-2.39 (m, 1H), 2.21-2.14 (m, 1H), 1.24-1.20 (m, 6H).

### Example 5: (2S)-isopropyl 2-{[((2′-deoxy-O⁶-methyl-guanosine)-5′-yloxy) (phenoxy) phosphoryl] amino} propanoate (2005)

### (2S)-isopropyl 2-{[(4-nitrophenyloxy) (phenoxy) phosphoryl] amino} propanoate (2001)

[chemical structure]

A solution of phenol (1.47 g, 15.62 mmol, 1 eq.) and anhydrous triethylamine (2.4 ml, 17.19 mmol, 1.1 eq.) in anhydrous dichloromethane (35 ml) was added dropwise to a solution of p-nitrophenyl phosphorodichloridate (4.00 g, 15.62 mmol, 1 eq.) in anhydrous dichloromethane (35 ml) under Argon atmosphere in a 250 ml round-bottom flask, cooled to −78° C. by means of dry ice/acetone bath. The resulting mixture was stirred at that temperature for 30 minutes and after that period, when ³¹P NMR confirmed completion of the reaction (CDCl₃, a singlet at −6.00 ppm corresponding to desired phosphorochloridate was observed), the reaction mixture was transferred through syringe to a 250 ml round-bottom flask containing a cold solution (0° C.) of L-alanine isopropyl ester hydrochloride (2.62 g, 15.62 mmol, 1 eq.) in anhydrous dichloromethane (35 ml). Subsequently, anhydrous triethylamine (4.6 ml, 32.82 mmol, 2.1 eq.) was added dropwise and the mixture was stirred at 0° C. for further 30 minutes. Once ³¹P NMR confirmed completion of the reaction (CDCl₃, appearance of doublet at −3.12 ppm), dichloromethane was evaporated under reduced pressure without any contact with air. The residue was suspended in diethyl ether and stirred at 0° C. for 30 minutes. The white solid was filtered off and the filtrate was concentrated under reduced pressure on rotary evaporator without any contact with air to obtain yellow oil of (2S)-isopropyl 2-{[(4-nitrophenyloxy) (phenyloxy) phosphoryl] amino} propanoate (1). C₁₈H₂₁N₂O₇P, M.W.: 408.35 g/mol; (6.12 g, 96%). ³¹P NMR (202 MHz, CDCl₃): δ 3.12 (d, J=3.7 Hz) ppm.

### 3′, 5′-di-O-acetyl-2′-deoxyguanosine (2002)

[chemical structure]

2′deoxyguanosine (10.00 g, 37.42 mmol, 1 eq.), 4-dimethylaminopyridine (0.46 g, 3.74 mmol, 0.1 eq.) and triethylamine (13.6 ml, 97.29 mmol, 2.6 eq.) were dissolved in anhydrous acetonitrile (500 ml) under Argon atmosphere in a 1000 ml round-bottom flask and the solution was cooled to 0° C. Acetic anhydride (8.5 ml, 89.81 mmol, 2.4 eq.) was added dropwise and the resulting reaction mixture was stirred overnight at room temperature. After addition of methanol (5 ml), the formed solid was filtered using a Büchner funnel and washed with methanol and hexane to obtain a white solid of 3′, 5′-di-O-acetyl-2′-deoxyguanosine (2). C₁₄H₁₇N₅O₆, M.W.: 351.32 g/mol; (12.60 g, 96%). ¹H NMR_(500 MHz, DMSO-d6): δ 10.67 (1H, s, NH), 7.92 (1H, s, H-8), 6.50 (2H, s, NH₂), 6.14 (1H, dd, J=8.8, 5.9 Hz, H-1′), 5.30 (1H, dt, J=6.2, 1.9 Hz, H-3′), 4.31-4.25 (1H, m, H-5′ a), 4.22-4.17 (2H, m, H-4′ and H-5′ b), 2.96-2.88 (1H, m, H-2′ a), 2.46 (1H, ddd, J=14.2, 6.0, 2.1 Hz, H-2′ b), 2.09 (3H, s, CH₃), 2.05 (3H, s, CH₃) ppm. Rf_{(DCM/MeOH, 9:1)}=0.47

### 3′, 5′-di-O-acetyl-6-deoxo-6-chloro-2′-deoxyguanosine (2003)

[chemical structure]

Compound 2002 (6.00 g, 17.08 mmol, 1 eq.) was suspended in anhydrous acetonitrile (100 ml) under Argon atmosphere in a 250 ml round-bottom flask together with benzyltriethylamonium chloride (5.83 g, 25.62 mmol, 1.5 eq.) and N,N-dimethylaniline (13.0 ml, 102.47 mmol, 6 eq.). The resulting mixture was cooled to 0° C. and phosphoryl chloride (9.6 ml, 102.47 mmol, 6 eq.) was added dropwise. The mixture was stirred for 10 minutes at room temperature and then heated to reflux in preheated oil bath. The reaction was monitored every 10 minutes by TLC (DCM/MeOH, 9:1) and after 1 hour, when there were no further changes observed on TLC plate, the reaction mixture was cooled with an ice bath and concentrated to dryness under reduced pressure. Ice water (20 ml) was added under cooling in order to hydrolyse the remaining phosphoryl chloride, the mixture was stirred for 20 minutes and subsequently extracted with ethyl acetate. The organic layers were joined, dried over sodium sulphate and the solvent was evaporated under reduced pressure on rotary evaporator. The crude residue was purified by column chromatography on silica using DCM/MeOH (95:5) as the eluting system yielding a white foam of 3′, 5′-di-O-acetyl-6-deoxo-6-chloro-2′-deoxyguanosine. C₁₄H₁₆ClN₅O₅, M.W.: 369.76 g/mol; (2.90 g, 46%). ¹H NMR (500 MHz, CDCl₃): δ 7.94 (1H, s, H-8), 6.31 (1H, dd, J=7.9, 6.2 Hz, H-1′), 5.45 (1H, dt, J=6.3, 2.5 Hz, H-3′), 5.21 (2H, s, NH₂), 4.48 (1H, dd, J=14.5, 6.01 Hz, H-5′ a), 4.41-4.36 (2H, m, H-4′ and H-5′ b), 3.00 (1H, ddd, J=14.2, 7.9, 6.4 Hz, H-2′ a), 2.59 (1H, ddd, J=14.2, 6.2, 2.6 Hz, H-2′ b), 2.16 (3H, s, CH₃), 2.11 (3H, s, CH₃) ppm. Rf_{(DCM/MeOH, 95:5)}=0.43.

### 2′-deoxy-O⁶-methyl-guanosine (2004)

[chemical structure]

Freshly prepared 1M solution of NaOCH₃ (5.84 g, 108.18 mmol, 5 eq.) in anhydrous MeOH (108.2 ml) was added dropwise to a solution of 2003 (8.00 g, 21.64 mmol, 1 eq.) in anhydrous methanol (50 ml) under Argon atmosphere in a 500 ml round-bottom flask, cooled to 0° C. The reaction mixture was stirred at room temperature for 6 hours, until no more starting material could be observed at TLC plate (DCM/MeOH, 9:1). The mixture was concentrated to dryness under reduced pressure, the residue was dissolved in small amount of water to obtain a transparent yellow solution. Acetic acid was used to adjust pH of the solution to pH 7, resulting in formation of a white solid of sodium acetate. Using decantation, the solid was extracted 5 times with ethyl acetate and 5 times with dichloromethane afterwards. The solid residue was then dissolved in as small amount of water as was necessary and extracted further two times with ethyl acetate and 2 times with dichloromethane. All organic layers were joined, dried over sodium sulphate and concentrated under reduced pressure on rotary evaporator yielding pure 2′-deoxy-O⁶-methyl-guanosine (4). C₁₄H₂₀O₁₀, M.W.: 281.27 g/mol; (4.21 g, 69%). ¹H NMR (500 MHz, MeOD): δ 8.05 (1H, s, H-8), 6.34 (1H, dd, J=8.3, 6.1 Hz, H-1′), 4.59-4.57 (1H, m, H-3′), 4.09-4.04 (4H, m, H-4′ and CH₃), 3.86 (1H, dd, J=12.2, 3.1 Hz, H-5′ b), 3.76 (1H, dd, J=12.2, 3.4H-5′ b), 2.84-2.76 (1H, m, H-2′ a), 2.36 (1H, ddd, J=13.4, 6.0, 2.6 Hz, H-2′ b) ppm. Rf_{(DCM/MeOH, 9:1)}=0.37.

### (2S)-isopropyl 2-{[((2′-deoxy-O⁶-methyl-guanosine)-5′-yloxy) (phenoxy) phosphoryl] amino} propanoate (2005)

[chemical structure]

Compound 2004 (1.30 g, 4.62 mmol, 1 eq.) was suspended in anhydrous DMF (35 ml) in a 150 ml round-bottom flask under Argon atmosphere, 1M tert-butylmagnesium chloride in THF (13.9 ml, 13.87 mmol, 3 eq.) was added dropwise and the resulting mixture was stirred for 30 minutes. A solution of 2001 (2.07 g, 5.08 mmol, 1.1 eq.) in anhydrous DMF (10 ml) was added dropwise over a period of 15 minutes and the reaction mixture was stirred for 48 hours at room temperature. Afterwards, the solvent was evaporated under reduced pressure on rotary evaporator and the residue was diluted with water resulting in formation of a solid that was filtered off. The water phase was extracted two times with dichloromethane and two times with ethyl acetate. Organic layers were joined, dried over sodium sulphate and concentrated under reduced pressure. The crude was purified by column chromatography on silica using DCM/MeOH (9:1) as the eluting system. Fractions containing the desired product were further purified by reversed phase column chromatography (C-18) using ACN/H₂O (90:10), 100% ACN in 50 minutes yielding pure (2S)-isopropyl-2-{[((2′-deoxy-O⁶-methyl-guanosine)-5′-yloxy) (phenyloxy) phosphoryl] amino} propanoate (2005) as a white solid. C₂₃H₃₁N₆O₈P, M.W.: 550.51 g/mol; (104 mg, 4%). ¹H NMR (500 MHz, MeOD): δ 7.98 (1H, s, H-8), 7.38-7.14 (5H, m, Ph), 6.37-6.32 (1H, m, H-1′), 4.99-4.90 (1H, m, OCH), 4.64-4.60 (1H, m, H-3′), 4.44-4.25 (2H, m, 2H-5′), 4.20-4.12 (1H, m, H-4′), 4.06 (3H, d, J=2.5 Hz, OCH₃), 3.93-3.83 (1H, m, NHCH) 2.86-2.71 (1H, m, H-2′ a), 2.44-2.37 (1H, m, H-2′ b), 1.32-1.26 (3H, m, NHCHCH₃), 1.23-1.18 (6H, m, CH₃CHCH₃) ppm. ¹³C NMR (125 MHz, MeOD): δ 173.24 (d, J=4.6 Hz)-172.99 (d, J=5.4 Hz)-161.23 (s)-160.42 (d, J=3.6 Hz)-153.19 (d, J=4.6 Hz)-150.74 (d, J=6.8 Hz) (6C, Ph-C1 and COO and C-2 and C-4 and C-5 and C-6), 138.04 (s)-137.84 (s) (1C, C-8), 129.53 (s)-129.36 (s)-125.16 (s)-124.74 (s)-120.20-119.95 (m) (5C, Ph-C2-C6), 85.38 (d, J=8.6 Hz)-85.26 (d, J=8.6 Hz) (1C, C-4′), 84.24 (1C, d, J=8.9 Hz, C-1′), 71.13 (1C, s, C-3′), 68.76 (1C, s, OCH), 66.35 (d, J=5.5 Hz)-66.15 (d, J=5.5 Hz) (1C, C-5′), 52.79 (1C, s, OCH₃), 50.37 (s)-50.24 (s) (1C, NHCH), 38.98 (s)-38.78 (s) (1C, C-2′), 20.57-20.45 (2C, m, CH₃CHCH₃), 19.10 (d, J=6.4 Hz)-18.94 (d, J=7.2 Hz) (1C, NHCHCH₃) ppm. ³¹P NMR (202 MHz, MeOD): δ 4.08 (s, 0.4P), 3.88 (s, 1P). HPLC: Rt=13.84, 14.05 min (gradient ACN/H₂O, 10:90, 100% ACN in 30 min, flow: 1 ml/min). MS (ESI+): m/z=551.20 [M+H]⁺. Rf_{(DCM/MeOH, 9:1)}=0.54.

### Example 6: Log P (pH 11.0) Assay and Caco-2 Permeability Assay

The Log P assay was performed according to a miniaturized 1-octanol/buffer shake flask method followed by LC/MS/MS analysis. Test compounds were prepared as 10 mM solutions dissolved in 100% DMSO. Test compounds (10 mM in DMSO; 2 μL/well) and QC samples (10 mM in DMSO; 2 μL/well) were transferred in duplicate from storage tubes to the 96-well polypropylene cluster tubes. Buffer was prepared as 80 mM phosphate, 80 mM borate, and 80 mM acetate solution at pH 11.0 with 1% DMSO. Buffer-saturated 1-octanol (149 μL/well) and 1-octanol saturated buffer (149 μL/well) were added to each well. Each of the tubes was vigorously mixed on their sides for 3 minutes and then shaken upright for 1 hour at a speed of 880 rpm at room temperature. The tubes were centrifuged at 2500 rpm for 2 minutes. The buffer layer sample was diluted by a factor of 20 and the 1-octanol layer was diluted by a factor of 200 with internal standard solution. Sample analysis was performed using a triple quadrupole mass spectrometer. Peak areas were corrected by dilution factors and by reference to an internal standard, and the ratio of the corrected peak areas were used to calculate the results (Log P value). Data Analysis—The Log P value for each compound was calculated using the following equation:

$$Log {D}_{oct \text{/} buffer} = log ( \frac{{[ 200 - fold dilution of compound ]}_{octanol} \times 200}{{[ 20 - fold compound ]}_{buffer} \times 20} )$$

The results are presented in Table 3.

The Caco-2 permeability assay was performed as follows. Caco-2 cells purchased from ATCC were seeded onto polyethylene membranes (PET) in 96-well BD Insert plates at 1×10⁵ cells/cm², and refreshed medium every 4-5 days until to the 21′ to 28^{th} day for confluent cell monolayer formation.

The transport buffer in the study was HBSS with 10 mM HEPES at pH 7.40±0.05. Test compounds were tested at 2 μM in the presence or absence of 30 μM novobiocin (BCRP inhibitor), verapamil (Pgp inhibitor), or GF120918 (BCRP/Pgp inhibitor) bi-directionally in duplicate. E3S control was tested at 5 μM in the presence or absence of efflux inhibitors bi-directionally in duplicate, while fenoterol and propranolol controls were tested at 2 μM in the absence of efflux inhibition in A to B direction in duplicate. Final DMSO concentration was adjusted to less than 1%. The plate was incubated for 120 minutes in a CO₂ incubator at 37±1° C., with 5% CO2 at saturated humidity without shaking. All samples were mixed with acetonitrile containing internal standard and centrifuged at 4000 rpm for 20 min. Subsequently, 100 μL supernatant solution was diluted with 100 μL distilled water for LC/MS/MS analysis. Concentrations of test and control compounds in starting solution, donor solution, and receiver solution were quantified by LC/MS/MS, using peak area ratio of analyte/internal standard, and permeation of lucifer yellow through the monolayer was measured to evaluate the cellular integrity.

The apparent permeability coefficient Papp (cm/s) was calculated using the equation:

Pₐₚₚ=(dCᵣ/dt)×Vᵣ/(A×C₀),

where dCᵣ/dt is the slope of the cumulative concentration of compound in the receiver chamber as a function of time (μM/s); Vᵣ is the solution volume in the receiver chamber (0.075 mL on the apical side, 0.25 mL on the basolateral side); A is the surface area for the transport, i.e., 0.0804 cm² for the area of the monolayer; and C₀ is the initial concentration in the donor chamber (μM).

Percent recovery was calculated using the equation:

% Recovery=100×[(Vᵣ×Cᵣ)+(V_{d}×C_{d})]/(V_{d}×C₀),

where V_{d} is the volume in the donor chambers (0.075 mL on the apical side, 0.25 mL on the basolateral side); and C_{d} and Cᵣ are the final concentrations of transport compound in donor and receiver chambers, respectively. The results are presented in Table 3.

TABLE 3
Log P and Caco-2 data for selected compounds

| | Log P | Caco-2 Pₐₚₚ |
|---|---|---|
| Compound | (@ pH = 11) | (A-B/B-A × 10⁻⁶)^{a} |
| dTMP | −1.9 | <0.03/<0.03 |
| 21 | 0.67 | 0.10/7.9 |
| 27 | 0.97 | 0.07/8.9 |
| 24 | 1.7 | 0.04/12.6 |
| dCMP | −2.1 | <0.04/<0.04 |
| 30 | 1.2 | 0.14/0.57 |
| 36 | 1.5 | 0.24/0.66 |
| 33 | 2.1 | 0.19/1.39 |
| dGMP | −1.4 | 0.28/0.51^{b} |
| 12 | −0.03 | 0.17/0.64 |
| 18 | 0.24 | 0.16/0.57 |
| 15 | 1.1 | 0.10/1.76 |
| 1017 | 3.0 | 2.0/5.7 |
| 1023 | 1.7 | 0.06/0.49 |
| 14 | 3.4 | 0.04/0.31 |

^{a}with efflux inhibitor,
^{b}recovery <2%

### Example 7: dNMP Prodrugs Rescue mtDNA Depletion in Patient-Derived Fibroblasts with DGUOK Deficiency

Patient-derived fibroblast cell line 10028 containing a DGUOK splicing variant c.592-4_c.592-3delTT and a c.677A>G (p.H226R) resulting in a severe neonatal onset hepatocerebral presentation and mtDNA depletion was used, as described in Buchaklian et al., Molecular Genetics and Metabolism, 2012, 107, 92-94. Cells were cultured in 3.5-cm diameter plates containing αMEM with 10% FBS plus 20 mM L-glutamine. Once confluent, cells were supplemented with serum-starved αMEM plus 20 mM L-glutamine. Compounds were dissolved in DMSO vehicle and added to media containing cells to give a final concentration between 1 and 100 μM. Control cells were supplemented with vehicle only. Cells were incubated with compound or vehicle for 10 consecutive days in serum-starved media, which was exchanged daily with identical media containing freshly prepared compound or vehicle. mtDNA copy number was assessed via qPCR as described in Venegas et al., Current Protocols in Human Genetics, 2011, Chapter 19, Unit 19.7. The results are presented in FIG. 1. Both of the dNMP prodrugs tested, compounds 15 and 1017, were found to increase mtDNA copy number relative to control in a dose-dependent manner.

### Example 8: Evaluation of Exemplary Compounds in Liver Slice Cultures

Liver slice culture was tested by extracting the liver, coring tissue, and slicing with the Krumdieck slicer ranging from 3-15 mm in diameter with a thickness of 250 microns. Primary hepatocyte culture was performed using liver perfusion via the hepatic portal vein. Collagenase buffer was utilized to dissociate hepatocytes through digestion. The liver was removed from the animal, cells were dispersed, counted and plated. mtDNA content was evaluated by real-time qPCR with two specific primer sets (one targeting the mitochondrial gene MT-TL1 and the other the nuclear gene beta-actin). Drug treatment of primary hepatocytes was performed using DMSO (negative control) and dGMP (positive control) in comparison to experimental agents 15, 1017, and 2005. Agents were added daily to primary liver cultures for up to 10 days following tissue collection. The results are presented in FIG. 2. Compound 2005 was superior to compounds 15 and 1017.

### Other Embodiments

All publications and patents mentioned herein are hereby incorporated by reference in their entirety as if each individual publication or patent was specifically and individually indicated to be incorporated by reference. In case of conflict, the present application, including any definitions herein, will control.

While specific embodiments of the subject invention have been discussed, the above specification is illustrative and not restrictive. Many variations of the invention will become apparent to one skilled in the pharmaceutical art upon review of this specification and the claims below. The full scope of the invention should be determined by reference to the claims, along with their full scope of equivalents, and the specification, along with such variations.

Having thus described in detail preferred embodiments of the invention, other embodiments will be evident to one skilled in the pharmaceutical art. The foregoing detailed description is provided for clarity only and is merely exemplary. The spirit and scope of the invention are not limited to the above examples, but are encompassed by the following claims.

We claim:

1. A compound having the structure of formula I or a pharmaceutically acceptable salt or prodrug thereof: [chemical structure]
wherein:
R¹ is selected from aryl and heteroaryl;
R² and R^{2′} are each independently is selected from hydrogen, alkyl, aralkyl, and a natural amino acid side chain;
R³ is selected from alkyl and aralkyl;
R⁴ is selected from hydrogen and alkyl; or R² and R⁴ together with the —C—N— moiety that separates them form a heterocycle; and
NT is selected from a nucleobase and a nucleobase prodrug moiety.

2. The compound of claim 1, wherein:
R¹ is selected from phenyl, naphthyl, and halo-phenyl; and
R⁴ is hydrogen.

3. The compound of claim 2, wherein
R¹ is selected from phenyl, naphthyl, and fluoro-phenyl.

4. The compound of claim 2, wherein
R¹ is selected from phenyl, naphthyl, and 4-fluoro-phenyl.

5. The compound of claim 2, wherein
R² is selected from alkyl and H; and
R², is selected from alkyl and H.

6. The compound of claim 4, wherein
R² and R^{2′} ⋅ are independently selected from alkyl and hydrogen.

7. A compound of claim 6, wherein:
R² is H; and
R^{2′} is methyl.

8. A compound of claim 2, wherein:
R³ is selected from alkyl, branched alkyl, and aralkyl.

9. A compound of claim 4, wherein:
R³ is selected from methyl, isopropyl, and benzyl.

10. A compound of claim 6, wherein:
R³ is selected from methyl, isopropyl, and benzyl.

11. A compound of claim 2, wherein:
NT is selected from adenine, guanine, cytosine, and thymine.

12. A compound of claim 4, wherein:
NT is selected from adenine, guanine, cytosine, and thymine.

13. A compound of claim 6, wherein:
NT is selected from adenine, guanine, cytosine, and thymine.

14. A compound of claim 10, wherein:
R³ is selected from methyl, isopropyl, and benzyl.

15. A compound of claim 2, wherein NT is selected from: [chemical structure]
wherein R¹¹ is amino; and R¹² is methyl.

16. A compound of claim 4, wherein NT is selected from: [chemical structure]
wherein R¹¹ is amino; and R¹² is methyl.

17. A compound of claim 6, wherein NT is selected from: [chemical structure]
wherein R¹¹ is amino; and R¹² is methyl.

18. The compound of claim 2, wherein the nucleobase prodrug moiety is selected from: [chemical structure]
wherein R⁵ is alkyl or aralkyl.

19. The compound of claim 11, wherein the nucleobase prodrug moiety is selected from: [chemical structure]
wherein R⁵ is selected from alkyl and aralkyl.

20. The compound of claim 12, wherein the nucleobase prodrug moiety is selected from: [chemical structure]
wherein R⁵ is selected from alkyl and aralkyl.

21. The compound of claim 1, wherein the compound is: [chemical structure]
or a pharmaceutically acceptable salt thereof.

22. The compound of claim 1, having the structure of formula Ia: [chemical structure]
or a pharmaceutically acceptable salt or prodrug thereof, wherein:
R¹ is selected from phenyl. naphthyl, and 4-fluorophenyl;
R² is methyl;
R³ is selected from methyl, isopropyl. and benzyl;
R⁴ is hydrogen; and
NT is selected from adenine, guanine, cytosine, and thymine.

23. The compound of claim 1, having the structure of formula II: [chemical structure]
or a pharmaceutically acceptable salt or prodrug thereof, wherein:
R¹ is selected from hydrogen and alkyl;
R^{2a} and R^{2b} are each independently selected from hydrogen, alkyl, aralkyl, and a natural amino acid side chain;
R³ is alkyl; and
NT is selected from a nucleobase and a nucleobase prodrug moiety.

24. A compound of claim 1, wherein the compound is selected from compounds listed in Table 1.

25-33. (canceled)
